(19) United States
(12) Patent Application Publication
(10) Pub. No.: US 2024/0342276 A1
(43) Pub. Date: Oct. 17, 2024

(54) SILICON DIOXIDE VACCINE DELIVERY SYSTEM TAKING VIRUS-LIKE PARTICLES AS TEMPLATE, AND CONSTRUCTION METHOD AND APPLICATION OF SILICON DIOXIDE VACCINE DELIVERY SYSTEM

(71) Applicant: DALIAN UNIVERSITY OF TECHNOLOGY, Dalian, Liaoning (CN)

(72) Inventors: Bingbing SUN, Dalian, Liaoning (CN); Min LI, Dalian, Liaoning (CN); Changying XUE, Dalian, Liaoning (CN); Zhihui LIANG, Dalian, Liaoning (CN); Lei ZHANG, Dalian, Liaoning (CN); Chen CHEN, Dalian, Liaoning (CN)

(21) Appl. No.: 18/293,608

(22) PCT Filed: Nov. 25, 2022
(86) PCT No.: PCT/CN2022/134208

§ 371 Date: Jan. 30, 2024

(30) Foreign Application Priority Data
Jan. 10, 2022 (CN) 202210023629.2

(51) Int. Cl. A61K 39/39; A61K 39/00; A61K 39/12; A61K 39/29; A61P 37/04

(52) U.S. Cl. CPC A61K 39/39; A61K 39/12; A61K 39/292; A61P 37/04; A61K 2039/5258; A61K 2039/55505

(57) ABSTRACT

A silicon dioxide vaccine delivery system uses virus-like particles as templates. The particle morphology of the silicon dioxide vaccine system is 50-500 nm of nanoparticles, of which an antigenic component is 20-200 nm of virus-like particles, an adjuvant component is nano silicon dioxide, the silicon dioxide component is wrapped on the surface of the virus-like particle, and a mass ratio of silicon element to antigen is 50-0.5:1. The construction of the silicon dioxide vaccine delivery system includes steps of: (1) adding a proper amount of 3-aminopropyltriethoxysilane into an aqueous solution containing virus-like particles and stirring; (2) adding a proper amount of tetraethoxysilane into the dispersion system in step (1) and stirring; and (3) centrifuging a reactant obtained in step (2) and removing a supernatant to obtain a product. A vaccine constructed by means of the vaccine system can trigger a host to generate humoral and cellular immune levels.

### TECHNICAL FIELD

The present disclosure relates to construction and application of silicon dioxide vaccine delivery system taking virus-like particles as a template, and particularly relates to a method for synthesizing silica nanoparticles using antigenic virus-like particles as a template, which can be applied in virus vaccines development for the prevention and treatment of various infectious diseases.

### BACKGROUND

Since 2019, a novel coronavirus has caused a global pneumonia outbreak. As of Dec. 6, 2021, nearly 270 million confirmed cases of COVID-19, including more than 5.25 million deaths, have been reported to the World Health Organization worldwide. Injection of preventive COVID-19 vaccines is one of the most efficient and convenient means for people to resist the new coronavirus disease. Nearly 4 billion doses of COVID-19 vaccines have been administered so far, greatly protecting the life and property safety of the world population.

As the most important weapon to prevent infectious diseases, vaccines have gone through several stages of development. In 1798, the British physician Jenner developed the world's first cowpox vaccine, marking the beginning of the vaccine application. Until the mid-to-late 20th century, the development of vaccines entered a golden age. As an essential component of vaccines, adjuvants play an extremely important role in enhancing the immune response to antigens. The development of adjuvants has gone through two stages: from natural ingredients to synthetic engineered vaccine adjuvants. The discovery of the adjuvant effect of aluminum salts in 1926 has epoch-making significance. So far, among the vaccines approved by the U.S. FDA, there are six types of adjuvants, including aluminum salt adjuvants, MF59, AS03, AS04, CpG ODN and AS01B. To be specific, the aluminum salts are particularly important in adjuvant vaccines and are widely used in vaccines for tetanus, diphtheria, pertussis, poliomyelitis, hepatitis A, hepatitis B, and the like.

However, in terms of the efficacy of vaccine adjuvants, simple aluminum salt adjuvants generally can only enhance the level of humoral immunity, but cannot improve the level of cellular immunity of the host. In addition, excipients are usually added to vaccine formulations to improve the interaction between antigens and adjuvants, which makes the vaccine production complicated. Therefore, it is of great significance of designing a vaccine with a simple production process capable of triggering a strong and balanced immune response in the prevention and treatment of infectious diseases.

### SUMMARY

The present disclosure constructs a silicon dioxide vaccine delivery system taking virus-like particles as a template. It can employ various virus-like particles as a template to prepare a virus vaccine with efficient immune activity by means of a simple and effective nano-silicon dioxide synthesis method. Further, it can construct vaccines based on various virus-like particles to prevent and treat the corresponding infectious diseases.

An objective of the present disclosure is to provide a silicon dioxide vaccine delivery system taking virus-like particles as a template, including virus-like particles and silicon dioxide, where the virus-like particles serve as antigens, the nano-silicon dioxide serves as an adjuvant component, and the silicon dioxide is coated on surfaces of the virus-like particles to form nanoparticles.

Further, the vaccine delivery system is nanoparticles, and the nanoparticles are preferably nanoparticles having morphology of 50-800 nm.

Further, types of the virus-like particles include but are not limited to common virus-like particles such as hepatitis B surface antigen virus-like particles, hepatitis B core antigen virus-like particles, human papillomavirus-like particles and novel coronavirus-like particles, as well as chimeric virus-like particles that take the hepatitis B core antigen virus-like particles as carriers to chime novel coronavirus receptor-binding domain proteins and take hepatitis B surface antigen virus-like particles as carriers to chime influenza virus antigens, and the like, and particle sizes of the virus-like particles are preferably 20-200 nm.

Another objective of the present disclosure is to provide an application of the silicon dioxide vaccine delivery system taking virus-like particles as a template in various preventive and therapeutic vaccines, which constructs vaccines such as hepatitis B vaccine, papillomavirus vaccine, new coronavirus vaccine, influenza virus vaccine and the like based on the above various virus-like particles.

Further, a mass ratio of silicon element to virus-like particles in the vaccine is 50-0.5:1, preferably 20-1:1.

Another objective of the present disclosure is to provide a method for constructing a silicon dioxide vaccine delivery system taking virus-like particles as a template, and the method includes the following steps of:

(1) adding a proper amount of 3-aminopropyltriethoxysilane into an aqueous solution containing certain virus-like particles, and stirring to obtain a dispersion system;

(2) adding a proper amount of tetraethoxysilane into the dispersion system obtained in the step (1), and stirring to obtain a reactant; and

(3) centrifuging the reactant obtained in the step (2) and removing a supernatant to obtain a vaccine product, and storing the vaccine product after centrifugally washing it with ultrapure water.

Further, a concentration of the virus-like particles in the step (1) in the reaction system is 0.01-10 mg/mL, preferably 0.2-10 mg/mL, and a concentration of 3-aminopropyltriethoxysilane is 0.1-100 mM, preferably 0.1-20 mM. A concentration of tetraethoxysilane in the reaction system in the step (2) is 0.1-500 mM, preferably 0.2-50 mM.

Further, a storage concentration of a vaccine synthesized in the step (3) is preferably 5-100 μg/mL for virus-like particles and 20-1000 μg/mL for silicon dioxide.

Further, a stirring speed in the step (1) is 300-1500 rpm, preferably 600-1200 rpm, and a stirring time is 10 s-30 min, preferably 30 s-20 min. A stirring speed in the step (2) is 300-1500 rpm, preferably 600-1200 rpm, and a stirring time is 30 min-30 h, preferably 2 h-24 h. Reaction temperatures in the steps (1) and (2) are 4-50° C., preferably 4-30° C.

The present disclosure has the beneficial effects as follows:

The silicon dioxide vaccine constructed by taking virus-like particles as a template in the present disclosure is capable of simultaneously inducing efficient humoral immunity and cellular immunity as verified by in-vivo experiments in mice.

In the present disclosure, the construction method of the silicon dioxide vaccine platform capable of simultaneously inducing efficient humoral immunity and cellular immunity is simple and easy to operate, has good repeatability and mild reaction conditions, and finally obtains vaccine nanoparticles evenly dispersed with uniform particle sizes and good stability, embracing bright application prospects in the prevention and treatment of infectious disease viruses.

### BRIEF DESCRIPTION OF THE DRAWINGS

The present disclosure is accompanied with 8 drawings.

FIG. 1 is a schematic diagram of a synthesis mechanism of a silicon dioxide vaccine (VLP@Silica) taking virus-like particles (VLP) as a template.

FIG. 2 is a transmission electron microscope (TEM) images of hepatitis B surface antigen virus-like particles (HBsAg VLP) and HBsAg VLP@Silica particles, where a scale of the left image is 100 nm and a scale of the right image is 150 nm.

FIG. 3 is a dark field scanning TEM image (A) and energy-dispersive X-ray spectroscopy distribution diagrams (B to D) of HBsAg VLP@Silica particles, where scales of the image and diagrams are all 200 nm; FIGS. 3B and 3C are distribution diagrams of Si and O elements respectively, and FIG. 3D is a combined diagram of distribution of Si and O elements.

FIG. 4 is an infrared spectrogram of HBsAg VLP and HBsAg VLP@Silica.

FIG. 5 shows the detection of hepatitis B antibody levels induced by HBsAg VLP@Silica vaccine using C57BL/6 mice aged 6-8 weeks as models, where FIG. 5A shows a hepatitis B antigen immunization strategy, in particular, the mice are intramuscularly injected with HBsAg VLP@Silica containing 2 μg hepatitis B surface antigens on day 0, the same amount of HBsAg VLP@Silica is injected into the mice on Day 14, serum and spleen of each mouse are taken on Day 28 to detect levels of humoral immunity and cellular immunity. FIGS. 5B to 5D show the levels of specific antibodies in serum, respectively, that is, levels of total IgG, IgG₁ and IgG_{2c}, and FIG. 5E shows a ratio of IgG_{2c}/IgG₁, where saline represents normal saline, HBsAg VLP represents pure hepatitis B surface antigens, and HBsAg VLP+Alum represents a mixture of HBsAg VLP and a commercial aluminum oxyhydroxide adjuvant.

FIG. 6 shows the detection of cytokine release from T cells induced by HBsAg VLP@Silica vaccine using C57BL/6 mice aged 6-8 weeks as models, in particular, FIGS. 6A and 6B show levels of IFN-γ and IL-4 secreted by CD4⁺ T cells 4, FIGS. 6C and 6D show levels of IFN-γ and IL-4 secreted by CD8⁺ T cells.

FIG. 7 is transmission electron microscope images of human papillomavirus-like particles (HPV VLP) and human papillomavirus-like particle silica vaccine (HPV VLP@Silica), where scales of the images are both 300 nm.

FIG. 8 shows the detection of human papillomavirus (HPV) antibody levels and T cell-mediated immune responses induced by HPV VLP@Silica using C57BL/6 mice aged 6-8 weeks as models. A specific immunization strategy of the HPV VLP@Silica vaccine is as follows: the mice are intramuscularly injected with HPV VLP@Silica containing 2 μg of HPV VLP on Day 0 and Day 21, respectively, and serum and spleen of each mouse are taken on Day 42 to detect levels of humoral immunity and cellular immunity. Specifically, FIGS. 8A to 8C show the levels of specific antibodies in serum, respectively, that is, levels of total IgG, IgG₁ and IgG_{2c}, FIG. 8D shows a ratio of IgG_{2c} to IgG₁, where HPV VLP is pure human papillomavirus antigens, and FIGS. 8E to 8F show the CD69 expression of CD4⁺ and CD8⁺ T cells after re-stimulation with 2 μg/mL HPV VLP in vitro.

### DETAILED DESCRIPTIONS OF THE EMBODIMENTS

The following non-limiting examples can make those of ordinary skill in the art better understand the present disclosure, but do not limit the present disclosure in any way.

### Example 1

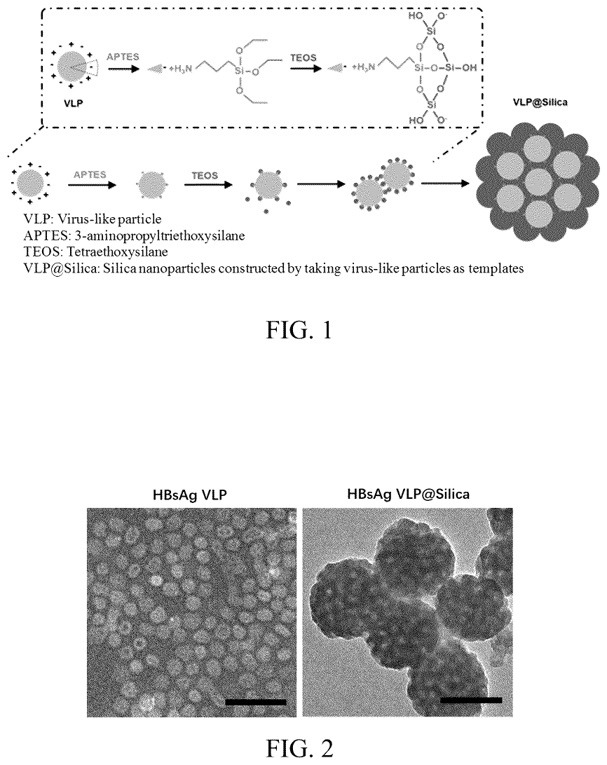

A method for constructing a silicon dioxide vaccine delivery system (HBsAg VLP@Silica) taking hepatitis B surface antigen virus-like particles (HBsAg VLP) as a template (FIG. 1), and the method includes the following steps:

(1) At room temperature, 3-aminopropyltriethoxysilane was added into an aqueous solution containing 2 mg/mL of HBsAg VLP to make the concentration of 3-aminopropyltriethoxysilane in the reaction system be of 50 mM, and the reaction system was then stirred at 800 rpm for 30 min;

(2) At room temperature, tetraethoxysilane was added into the reaction system in the step (1) to make its concentration in the reaction system be of 500 mM, and the solution was then stirred at 800 rpm for 30 h; and

(3) A reactant obtained in the step (2) was centrifuged and a supernatant therein was removed to obtain a product, the product was centrifugally washed three times with ultrapure water, and then was stored in normal saline, where a concentration of virus-like particles during storage was 40 μg/mL.

### Example 2

Detection of physical and chemical properties of the silicon dioxide vaccine (HBsAg VLP@Silica) taking hepatitis B surface antigen virus-like particles (HBsAg VLP) as a template prepared in Example 1 and the hepatitis B surface antigen virus-like particles (HBsAg VLP).

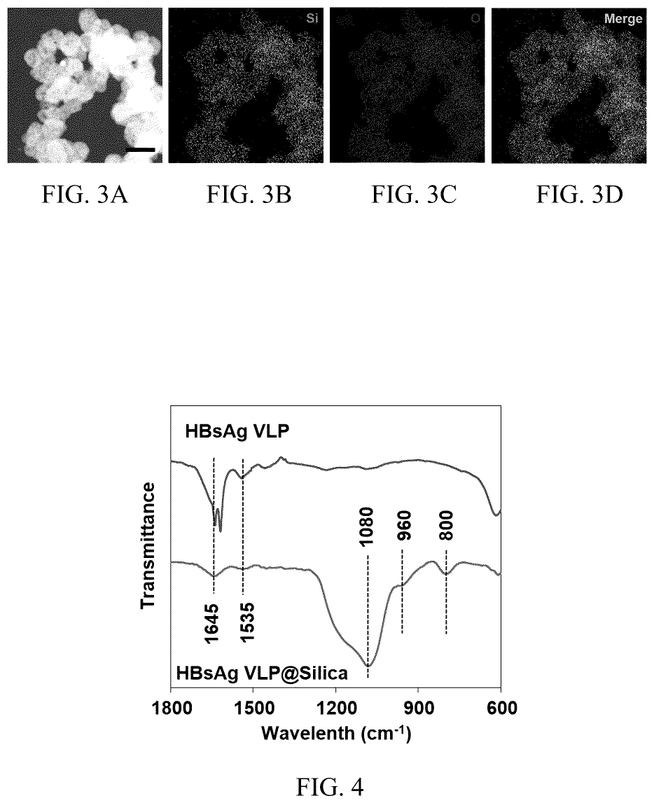

Morphology of the product (as shown in FIG. 2) obtained in Example 1 was detected through a transmission electron microscope (TEM), showing that the HBsAg VLP@Silica was raspberry-like nanoparticles of 137±19 nm. Element distributions of Si and O in the product (as shown in FIG. 3) were detected through dark field scanning TEM and energy-dispersive X-ray spectroscopy, showing the existence of silica in the HBsAg VLP@Silica. Functional groups of the HBsAg VLP@Silica were analyzed by infrared spectrum. As shown in FIG. 4 wavelengths of 1645 cm⁻¹ and 1550 cm⁻¹ represent amide I and II bands of HBsAg VLP, respectively, wavelengths of 1080 and 800 cm⁻¹ represent asymmetric and symmetric stretching vibration peaks of Si—O—Si in silica, respectively, and wavelength of 960 cm⁻¹ represents stretching vibration peak of Si—OH in silica, indicating that the HBsAg VLP@Silica was successfully prepared. An actual particle size of HBsAg VLP@Silica particles was calculated, a hydrated particle size and Zeta potential of the product were detected through a particle size analyzer, a content of HBsAg VLP in the supernatant of the reactant was detected through a BCA experiment, and a content of HBsAg VLP in HBsAg VLP@Silica was then calculated. Further, a content of Si element in the product was calculated through an inductively coupled plasma (ICP) spectroscopy, and a mass ratio of Si to HBsAg VLP in the product was finally obtained (Table 1).

Actual particle size, hydrated particle size, and Zeta potential of HBsAg VLP and HBsAg VLP@Silica and mass ratio of Si to HBsAg VLP are shown in Table 1.

TABLE 1

| | | Hydrated | Zeta | |
|---|---|---|---|---|
| | Size | particle size | potential | Si:HBsAg |
| Sample | (nm) | (nm) | (mV) | (w/w) |
| HBsAg VLPs | 22 ± 1 | 103 ± 5 | −15 ± 2 | — |
| HBsAg VLP@Silica | 137 ± 19 | 275 ± 4 | 31 ± 1 | 9.72 ± 0.40 |

### Example 3

C57BL/6 mice aged 6-8 weeks were used as animal models to detect levels of humoral immunity and cellular immunity of the HBsAg VLP@Silica prepared in Example 1, and the detection method included the following steps: the mice were intramuscularly injected with HBsAg VLP@Silica (50 μL normal saline containing 2 μg hepatitis B surface antigens and 20 μg silicon element) on Day 0, the same amount of HBsAg VLP@Silica was injected into the mice on Day 14, serum and spleen of each mouse was taken on Day 28 to detect levels of total IgG, IgG₁ and IgG_{2c} in serum, as well as the maturation and differentiation of splenocytes and the ability to secrete cytokines. In addition, control groups were introduced: normal saline group (each mouse was injected with 50 μL of normal saline), HBsAg VLP group (each mouse was injected with 2 μg of pure hepatitis B surface antigens), and HBsAg VLP+Alum group (each mouse was injected with a mixture of 2 μg HBsAg VLP and a commercial aluminum oxyhydroxide adjuvant (Alhydrogel® adjuvant 2%, InvivoGen) containing 20 μg aluminum element). Each group had seven experimental mice.

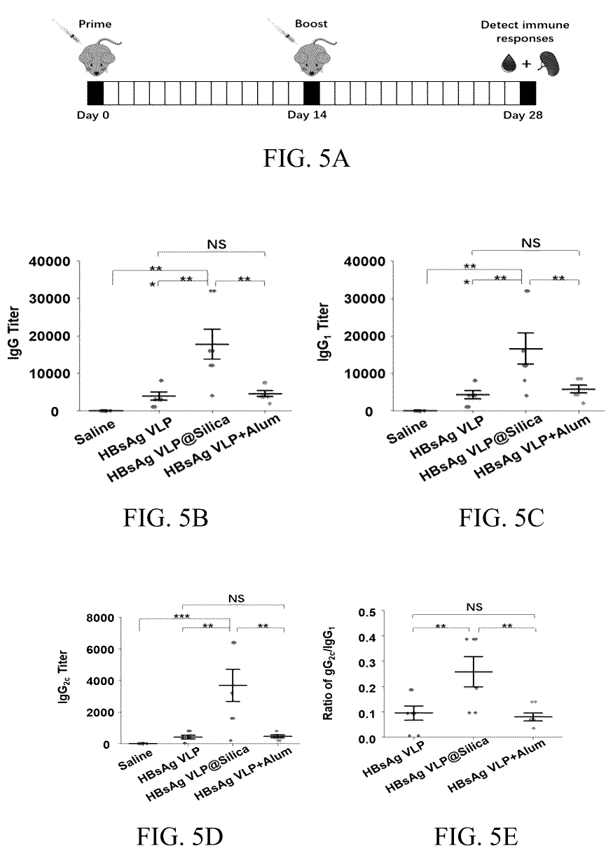

Levels of the total IgG, IgG₁ and IgG_{2c} in the serum, as well as ratio of IgG_{2c} to IgG₁ are shown in FIG. 5.

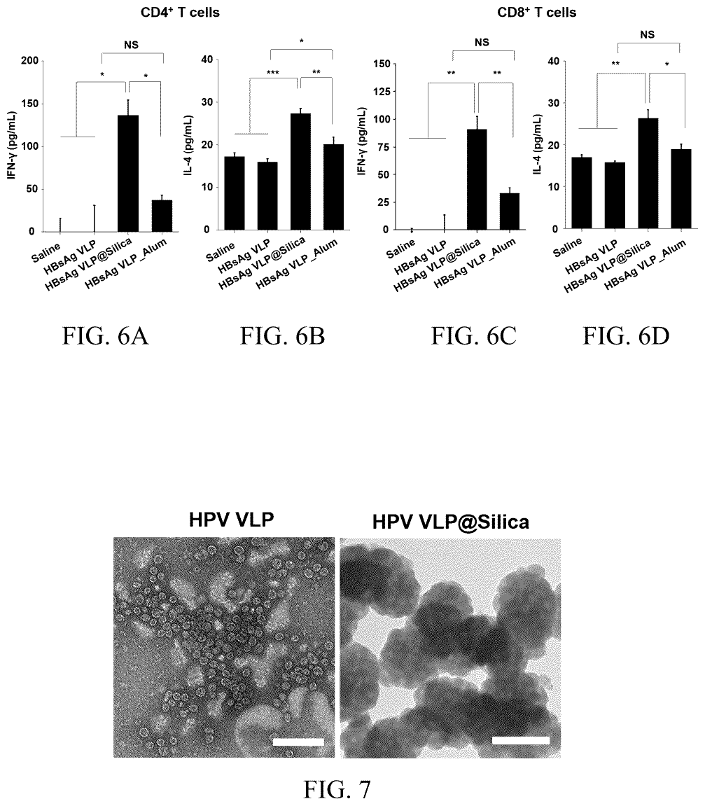

The maturation and differentiation of splenocytes and the ability to secrete cytokines are shown in FIG. 6.

As shown in FIGS. 5 and 6, according to characterization results of Example 3, results of antibody titer experiments show that the IgG, IgG₁ and IgG_{2c} antibody titers produced by HBsAg VLP@Silica are higher than those of HBsAg VLP and HBsAg VLP+Alum, and the ratio of IgG_{2c} to IgG₁ is higher, proving that the HBsAg VLP@Silica can produce more balanced levels of humoral immunity and cellular immunity. Results of cytokine secretion show that HBsAg VLP@Silica is capable of inducing CD4⁺ T cells and CD8⁺ T cells to secrete higher levels of IFN-γ and IL-4, indicating that it is capable of inducing a stronger cell-mediated immune response. In conclusion, the silicon dioxide vaccine delivery system (HBsAg VLP@Silica) taking HBsAg VLP as a template is capable of inducing stronger humoral and cellular immune responses.

### Example 4

A human papillomavirus-like particle silica vaccine (HPV VLP@Silica) was constructed through the silica delivery system construction method. The specific synthesis process was the same as that in Example 1, but HBsAg VLP was replaced by HPV VLP. HPV VLP and HPV VLP@Silica images are shown in FIG. 7, showing that the HPV VLP@Silica was raspberry-like nanoparticles of 350±20 nm.

C57BL/6 mice aged 6-8 weeks were used as animal models to detect a level of immunity induced by the human papillomavirus-like particle silica vaccine (HPV VLP@Silica), and the detection method included the following steps:

The mice were intramuscularly injected with HPV VLP@Silica (50 μL normal saline containing 2 μg human papillomavirus-like particles and 40 μg silicon element) on Day 0, the same amount of HPV VLP@Silica was injected into the mice on Day 14, serum and spleen of each mouse was taken on Day 28 to detect levels of total IgG, IgG₁ and IgG_{2c} in serum. In addition, a control group was introduced: HPV VLP group (each mouse was injected with 2 μg of pure human papillomavirus-like particles). Each group had seven experimental mice.

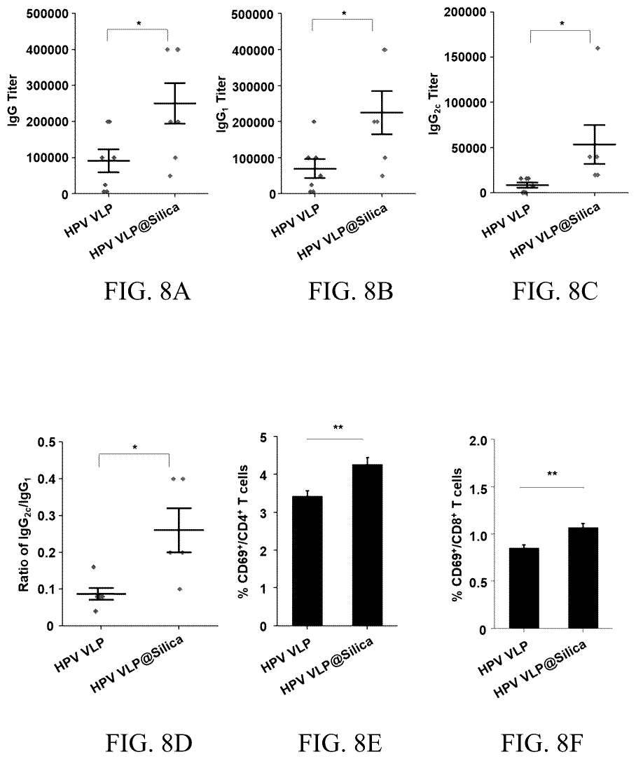

Levels of the total IgG, IgG₁ and IgG_{2c} in the serum, as well as ratio of IgG_{2c} to IgG₁ are shown in FIGS. 8A to 8D. Activation of CD4+ and CD8+ T cells is shown in FIGS. 8E to 8F.

As shown in FIG. 8, according to characterization results of Example 4, results of antibody titer experiments show that the IgG, IgG₁ and IgG_{2c} antibody titers produced by HPV VLP@Silica are higher than those of HPV VLP, and the ratio of IgG_{2c} to IgG₁ is higher, proving that the HPV VLP@Silica can produce more balanced levels of humoral immunity and cellular immunity. Results of T cells activation show that HPV VLP@Silica is capable of inducing higher expression of CD69 on surfaces of the CD4⁺ T and CD8⁺ T cells, indicating that it is capable of inducing the activation of T cells. In conclusion, the silicon dioxide vaccine delivery system (HPV VLP@Silica) taking HPV VLP as a template is capable of inducing stronger humoral and cellular immune responses.

Both vaccines (HBsAg VLP@Silica and HPV VLP@Silica) constructed through the above vaccine system of the present invention have verified that the vaccine system can induce the host to generate more powerful and balanced levels of humoral immunity and cellular immunity.

1. A silicon dioxide vaccine delivery system taking virus-like particles as a template, comprising virus-like particles and silicon dioxide, wherein the virus-like particles serve as antigens, the silicon dioxide serves as an adjuvant component, and the silicon dioxide is coated on surfaces of the virus-like particles to form nanoparticles.

2. The silicon dioxide vaccine delivery system taking virus-like particles as a template according to claim 1, wherein the virus-like particles comprise common virus-like particles and chimeric virus-like particles.

3. The silicon dioxide vaccine delivery system taking virus-like particles as a template according to claim 1, wherein the nanoparticles are nanoparticles having morphology of 50-800 nm.

4. The silicon dioxide vaccine delivery system taking virus-like particles as a template according to claim 1, wherein a mass ratio of silicon element to virus-like particles is 50-0.5:1.

5. The silicon dioxide vaccine delivery system taking virus-like particles as a template according to claim 2, wherein a particle size of the virus-like particles is 20-200 nm.

6. An application of the silicon dioxide vaccine delivery system constructed by taking virus-like particles as a template according to claim 1 in various preventive and therapeutic vaccines

7. A method for constructing the silicon dioxide vaccine delivery system constructed by taking virus-like particles as a template according to claim 1, comprising the following steps of:
(1) adding 3-aminopropyltriethoxysilane into an aqueous solution containing virus-like particles, and stirring to obtain a dispersion system;
(2) adding tetraethoxysilane into the dispersion system obtained in the step (1), and stirring to obtain a reactant; and
(3) centrifuging the reactant obtained in the step (2) and removing a supernatant to obtain a product, and storing the product after centrifugally washing it with ultrapure water.

8. The method for constructing the silicon dioxide vaccine delivery system constructed by taking virus-like particles as a template according to claim 7, wherein, in the step (1), a concentration of the virus-like particles in the reaction system is 0.01-10 mg/mL, a concentration of 3-aminopropyltriethoxysilane is 0.1-100 mM; and in the step (2), a concentration of tetraethoxysilane in the reaction system is 0.1-500 mM.

9. The method for constructing the silicon dioxide vaccine delivery system constructed by taking virus-like particles as a template according to claim 7, wherein in the step (3), a storage concentration of a vaccine synthesized is 5-100 μg/mL for virus-like particles and 0.02-20 mg/mL for silicon dioxide.

10. The method for constructing the silicon dioxide vaccine delivery system constructed by taking virus-like particles as a template according to claim 7, wherein a stirring speed in the step (1) is 300-1500 rpm and stirring time is 10 s-30 min, a stirring speed in the step (2) is 300-1500 rpm and stirring time is 30 min-30 h, and reaction temperatures in the steps (1) and (2) are 4-50° C.
